(19) United States
(12) Patent Application Publication
(10) Pub. No.: US 2021/0019655 A1
(43) Pub. Date: Jan. 21, 2021

(54) Equation Learning Neural Networks in Degrading Channels for Neural Machine Maintenance and Applications Thereof

(71) Applicant: Nanoscope Technologies, LLC, Bedford, TX (US)

(72) Inventors: Samarendra Kumar Mohanty, Arlington, TX (US); Sourajit Mitra Mustafi, Bedford, TX (US)

(21) Appl. No.: 16/933,527

(22) Filed: Jul. 20, 2020

Related U.S. Application Data
(60) Provisional application No. 62/876,587, filed on Jul. 19, 2019.

(51) Int. Cl. G06N 20/00; G16H 50/20; G16H 50/30

(52) U.S. Cl. CPC G06N 20/00; G16H 50/30; G16H 50/20

(57) ABSTRACT

The present invention generally relates to the method for evaluating a mathematical functional relationship of the variables and to applications of Equation Learning (EQL) Network in disease detection, diagnosis and screening. The present invention also relates to the data compression feature of EQL in effective communication using low power and bandwidth with applications in biotelemetry and satellite communication. The present invention also relates to the extrapolation capability of EQL in various diverse fields, including but not limited to neuro-prosthetics, stock/consumer market, navigation, power distribution, disease detection, environment protection and disaster management.

### CROSS-REFERENCE

Some references, which may include publications, patents, and patent applications, are cited and discussed in the description of this invention. The citation and/or discussion of such references is provided merely to clarify the description of the present invention and is not an admission that any such reference is “prior art” to the invention described herein. Citation of references in this application is by way of one or more numbers in parenthesis, which refer to the references listed at the end of the specification. All references cited and discussed in this specification are incorporated herein by reference in their entireties and to the same extent as if each reference were individually incorporated by reference.

This application claims the benefit of priority to U.S. Provisional Application No. 62/876,587 filed on Jul. 19, 2019, entitled “Equation Learning Neural Networks in Degrading Channels for Neural Machine Maintenance and Applications Thereof”, which is hereby incorporated by reference in its entirety.

### STATEMENT REGARDING FEDERALLY SPONSORED RESEARCH OR DEVELOPMENT

This invention was made primarily with funding by NanoScope Technologies, LLC. The Government has no rights in the invention.

### FIELD OF THE INVENTION

The present invention relates to the application of an Equation Learning Network (EQL) in degrading neural interface to accurately reconstruct the lost signal. EQL is a unique method of Artificial Intelligence (AI) learning technique that provides accurate data extrapolation and compression.

The present invention predominantly aims to aid people with neuro-prosthetics; the EQL increases the longevity of the neural interfaces manifold by compensating for data loss due to faulty channels.

### BACKGROUND OF THE INVENTION

Artificial Neural Networks are often said to be sensitive and critical with respect to extrapolation (1,2). Often, it is difficult to predict their behavior in case of data not having been part of the training data set (3-5). The prediction of a neural network is closely based on the data that has been used to train them. In order to obtain predictability and data extrapolation of neural networks in biological interfaces, the inventors introduced an equation learning neural network (EQL) (2) in a biological system to determine the underlying mathematical equation governing the training data. Data interpolation could thus be achieved robustly via many-to-one mapping using EQL.

Previously, Radial Based Functional (RBF) networks were used to extrapolate the data set; however, RBF has issues regarding accuracy in prediction (7,8). Causal learning, which aims at identifying a causal relationship between multiple observable, is another method for extrapolation. Although causal learning provides a factorization of the problem and reveals causes as well as effects, it leaves the exact functional dependency unexplained (6). Domain adaptation has similarity with EQL neural network (5). However, equation learning neural network differs from domain adaptation because training and exploration domains can operate completely independent of each other in EQL.

Brain-computer interfaces (BCI, or brain-machine interfaces) translate noisy neural activity into commands for controlling an effector via a decoding algorithm. In practice, decoders are successful at leveraging the statistical relationship between the intended movements of the user and firing rates of recorded neural signals (9). Under the operational assumption that some key variables of interest (e.g. effector kinematics) are linearly encoded by neural activities, the Kalman filter (KF) is a reasonable decoding approach, which empirically yields state-of-the-art decoding performance (10). However, for motion related prosthetics, the KF still relies on rotated velocities, and does not address the key issue of extending these insights to more complex tasks, such as control with a realistic multi joint arm effector. For example, in case of a simple arm movement (having 26 degrees of freedom), KF intentional based neural network suffers from intention mismatch corresponding to random noise applied to the user intention.

In case of visual prosthetics, there are still many open technological and biological challenges that need to be resolved. The patients implanted with state-of-the-art ARGUS II and Alpha IMS systems (11) are having visual acuity far from that required recognizing letters. Most of these visual prostheses have a very limited number of electrodes as compared to the number of cells in the retina, or visual cortex. For visual prosthetics, the problem is how to compress a large image of visual field (captured by camera) to retina or visual cortex without losing significant useful information.

The extrapolation neural networks can be best overcome with the application of our proposed Equation Learning Network (EQL) algorithm. The present invention describes the algorithm which gives the neural networks self-learning ability and aids both in data compression and extrapolation. The algorithm is not only important for maintenance of the neural interface, but also has versatile usefulness in temporal projection of real-life variables. The algorithm can also be used in image segmentation and analysis.

### SUMMARY OF THE INVENTION

The main objective of the invention is to provide a method based on an AI algorithm which can compensate for data loss in faulty and aging neural interfaces and completely reconstructs the lost data. The algorithm completely reconstructs data from a noisy channel. The equation learning (EQL) neural networks enhance the efficiency of neural interfaces, overcoming the challenges of faulty connections due to biological occurrences (e.g. scar tissue formation) or electrical noise in neural prosthetics.

In another embodiment, the present invention, provides the EQL Network which also aids in interpolation of the data. The network architecture also can compress the data via a many-to-one mapping procedure. For example, it can compress a large amount of data without loss of information. In addition, it can also reduce the redundancy in data.

Another feature of the present invention is that the EQL algorithm can evaluate a mathematical functional relationship of the variables; for example: arms movement with motor neuron response. The EQL algorithm uses these functional relationships to extrapolate data in a domain which is very different from the training data set. The EQL network also integrates conditional probability by predicting the most probable response for a stimulus. For data interpolation, it further uses many-to-one function to compress the data and reduce redundancy.

### BRIEF DESCRIPTION OF THE DRAWINGS

FIG. 1A and FIG. 1B detail schematics of extrapolation and data compression via an EQL network. FIG. 1A shows basic network architecture of equation learning (EQL) neural network with N^{th} iteration layers. The learning network follows N layers. There is a tradeoff between the number of layers added, the computation and accuracy of extrapolation. FIG. 1B shows the extrapolation and compression pathway of EQL network patterns.

FIG. 2 shows re-construction of cortical activities pattern using proposed EQL neural network in case of reduced field of view (FOV). The active neurons in the original image profile are labeled as blue crosses. The inventors subsequently reduced the FOV to 80, 60 and 30% respectively. Decrease in the FOV results in disappearance of spiking neurons, which are accurately reconstructed by EQL neural networks. The signals, which are reconstructed in 80%, 60% and 30% FOV are denoted by red, green and yellow crosses respectively.

FIG. 3 shows the accuracy of reconstructing neural activity data with our EQL model.

Extrapolation capability of EQL>95% even with 25% of original data available.

FIG. 4 shows reconstruction of cortical activities pattern using proposed EQL neural network in case of additional noise (Gaussian). The spiking neurons in the original image are labeled as blue crosses. Decrease of signal to noise ratio (SNR) in each subsequent step results in disappearance of signal from some neurons, which are correctly reconstructed by EQL networks. The neuronal activities that are reconstructed by EQL in case of 25, 50 and 75% SNR reduction are denoted by red, yellow and green crosses respectively.

FIG. 5 shows performance of the neural network with respect to noise addition (shown in red in X-axis) and data coverage. This simulation is conducted, in which the combined effect of high impedance channels (SNR decrease) and disruptive channels (loss in data coverage) is studied.

FIG. 6 shows performance of the EQL and Convolution Neural Network (CNN) with respect to various denoising methods. Gaussian, Laplace and LPCA (Local Principal Component Analysis) filters were used to denoise the data. SNR decrease is plotted in the X-axis, which relates to noise addition. The denoising results in signal smoothening. Even with 75% SNR reduction, the EQL predicts an accuracy>95% for all three types of denoising filter.

FIG. 7 shows an online experiment showing power of an EQL network for complete motion-signal reconstruction. Left: A mouse cursor is moved towards a fixed target in three different ways. The projected patterns are depicted in 10×10 array. The predicted pattern in EQL showing all the correctly reproduced pattern when all arrays are present. Right: Subsequent reduction in array size and extrapolated array signals (translucent area) are shown.

FIG. 8 shows reconstruction of electrical activity pattern in a 32-channel array using EQL algorithm. (Left) Electrical data generated from a stimulus. (Right) Even after presence of 50% active channels, the whole data pattern could be reconstructed using EQL network. The translucent area in the array represents reconstructed signals from EQL network pattern.

Table 1: Performance of Equation Learning Network (EQL) and Radial Basis function

(RBF) for data reconstruction.

Table 2: Performance of the Equation Learning Network (EQL) for noisy data and comparison with a convolution neural network (CNN).

### DETAILED DESCRIPTION OF INVENTION

Artificial Neural Networks are often said to be sensitive and critical with respect to extrapolation. Often it is hard to predict their behavior in case of data not having been part of the training data set (1). The prediction of neural network is closely based on the data that has been used to train them. In order to obtain predictability and data extrapolation of neural networks in biological interfaces, the inventors introduced an equation learning (EQL) neural network (2) in a biological system to determine the underlying mathematical equation governing the training data. Data interpolation could thus be achieved robustly via many-to-one mapping using EQL.

The present inventions describe a method to use EQL in neural interface, which is an algorithm that can extrapolate and interpolate real-life data.

An embodiment of the invention provides an AI algorithm which can compensate for data loss in faulty and aging neural interfaces and completely reconstructs the lost data. The algorithm completely reconstructs data from a noisy channel. The equation learning (EQL) neural networks enhance the efficiency of neural interfaces, overcoming the challenges of faulty connections due to biological occurrences (e.g. scar tissue formation) or electrical noise in neural prosthetics (11).

In another embodiment, the present invention, provides the EQL Network which also aids in the interpolation of the data. The network architecture also can compress the data via many-to-one mapping procedure. It can compress a large amount of data without loss of information. In addition, it can also reduce the redundancy in data.

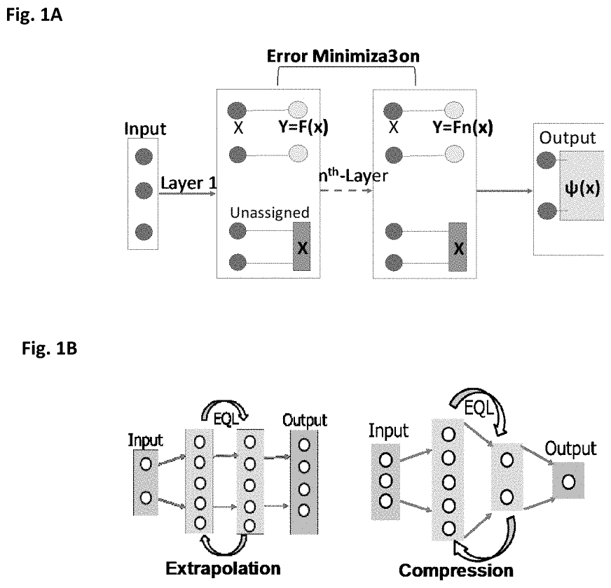

Another objective of the present invention is to evaluate a mathematical functional relationship of the variables; for example, arms movement and motor neuron response; and then uses these functional relationships to extrapolate data in the domain, which is very different from training data set. The EQL network also integrates conditional probability by predicting the most probable response for a stimulus. For data interpolation it further uses many-to-one function to compress the data and reduce redundancy. More detail is provided in FIG. 1A and 1B. They give detail schematics of extrapolation and data compression via EQL network.

In yet another embodiment of present invention, the EQL compensates for the degrading channels, thus increasing the longevity of neural interfaces via effective neural machine maintenance and benefit prosthetic-dependent injured personnel. For example, the EQL method according to the present invention will directly impact in developing an optogenetic cortical interface system for restoring vision lost in patients due to combat ocular trauma (COT) or traumatic optic neuropathy (TON) (12-15).

Another objective of the present invention is to develop a robust EQL algorithm for signal reconstruction in lossy neural interfaces (16-19). In another embodiment, a first step of the invention the identification of the underlying equation governing the relationship between external stimuli and neural response in neuro-prosthetic channels, which aids both data extrapolation as well as artificial signal stimulation.

In yet another embodiment, the present invention's AI code can generate simulated artificial signals that can be convoluted with original neural signals to increase the information content. Further, in an implantable device, often the recorded prosthetic data is too large to be effectively transmitted in low power mode.

Another objective of the present invention is to reduce redundancy. EQL network effectively compress the data. By reducing number of neural-nodes in the final layer of the AI step, EQL can effectively achieve data compression via many-to-one mapping.

In another embodiment, the equation learning (EQL) neural network of present invention, approaches data extrapolation and interpolation differently. First, it determines the functional dependence between neural activities and basic motion, like rotation of joint, finger motion etc. Then, it implements this basic function as a template to define complex motion. Complex motion is initially considered as a linear combination of simple motions. Later, in subsequent iteration steps, nonlinearity is introduced. Analytical characterization of complex motion significantly enhances the accuracy of the EQL algorithm according to the present invention. This gives EQL algorithm unique self-learning ability.

In another embodiment, EQL of the present invention has remarkable ability to predict and extrapolate any kind of stimuli, which are completely different from the training data set. For example, abnormal and uncontrolled arm movements for person suffering from neurodegenerative disease can be accurately characterized by EQL algorithm.

In an embodiment, the present invention's approach defines the correlation in a simple basic response like a simple joint movement in the forearm with respect to the signal in the electrode sensor as a response.

Eqⁿ 1: I₁=Σₙaₙf(x)ₙ, where summation over all sensors is performed. Moving a step forward, a slightly complex full arm motion is characterized by C=ΣbₘIₘ+ΣdₘIₘ²+Σⁿₘf_{m,n}IₘIₙ(Eqⁿ 2), where Iₘ is m^{th} simple response basis set. The first term in Eqₙ 2 is a linear term, second term is auto-correlation (dₘ is auto-correlation coefficient), and the third term is for cross-correlation (f_{m,n} is cross-correlation coefficient). While Eqₙ 2 is a second order complex response, invention can include but not limited to generating higher order of complex stimuli by increasing auto and cross-correlation terms.

In yet another embodiment, the present invention's analytical equation is based on simple response basis set (Iₘ), and very complex responses beyond the domain of training data are accurately extrapolated.

In another embodiment of the present invention for EQL extrapolation, the conditional probability distribution based on most probable sequence mode of action is imposed. For example, in case of arm movement some sequence of event is more probable than another, and some sequence of motion is impossible because of physical restriction.

Another objective of the present invention is therefore, by imposing certain probability and physical restriction, the extrapolation search space becomes narrow that significantly reduces the computational time.

In another embodiment, the present invention provides a method wherein the use of an EQL accurately reconstructs data from faulty channels. With only 10% of channels functioning, the EQL is able to reconstruct the whole signal with 95% accuracy.

In another embodiment, the present invention provides a method wherein the use of an EQL will increase the longevity of neuro-prosthetics by data compression and extrapolation.

In yet another embodiment, the present invention provides an algorithm wherein the EQL network can compress any signal in the range of 10,000 folds. It will efficiently transmit data through the neural interfaces with much less power consumption.

In another embodiment, the present invention provides an algorithm wherein the EQL networks compression will find application in the field of bio-telemetry.

In another embodiment, the present invention provides an algorithm wherein the EQL algorithm finds applications in longevity of network for people with artificial arms/legs and neuronal interfaces controlling their motions.

In another embodiment, the present invention provides a method wherein the use of an EQL will find applications for people with neuro-degenerative disease such as Parkinson's. The EQL algorithm integrated in neural interface can characterize the uncoordinated motion and give stimulation feedback to correct them.

In another embodiment, the present invention provides a method wherein the use of an EQL finds application in pain management therapeutics. EQL can act via neural interfaces to characterize and generate feedback to reduce pain.

In another embodiment, the present invention provides a method wherein the use of an EQL will find applications in people with impaired memory. For example, Alzheimer's patients, where there is progressive loss of memory, the EQL algorithm will recognize the signal pattern for various memory recollection and help in compensation of memory loss.

In yet another embodiment, the present invention provides a method wherein the use of EQL features will employ the unique combination of image recognition-based feature extraction (shape, size). For a relatively small amount of training data the EQL network will be able to classify various aspect of image recognition.

In another embodiment, the present invention provides a method wherein the use of an EQL will find applications in disease detection including various groups of blood cancer (hematological malignancies) including but not limited to Acute lymphoblastic leukemia, Acute myelogenous leukemia, Chronic lymphocytic leukemia, Chronic myelogenous leukemia, Acute monocytic leukemia, Hodgkin's lymphomas and Non-Hodgkin's lymphomas.

In yet another embodiment, the present invention provides a method wherein the use of an EQL will find application in analysis of the rate of blood flow in the heart valves.

In another embodiment, the present invention provides a method wherein the use of an EQL will find applications in prediction capability to determine the probability of stroke, arrhythmia in people.

In another embodiment, the present invention provides a method wherein the use of an EQL will find applications in detecting abnormalities in shapes and size of red blood cells and diseases associated with it. This can include but not limited to sickle cell anemia, and thalassemia.

In another embodiment, the present invention provides a method wherein the use of an EQL performs efficiently in image detection, segmentation and classification. The algorithm can be used to detect pathogenic bacteria and microbes present in the blood samples and can be used in disease detection.

In yet another embodiment, the present invention provides a method wherein the use of an EQL could also be trained to identify various type of toxic oligomers and fibrils in cerebrospinal fluid (CSF) and can effectively detect and diagnose various neurodegenerative diseases like Alzheimer's, Parkinson and Huntington.

In another embodiment, the present invention provides a method wherein the use of an EQL's capability of efficient image segmentation and classification could measure the pollutant population in sea and fresh water system and will be excellent in determining oil spills and its effect on environment.

In another embodiment, the present invention provides a method wherein the use of an EQL will find applications in performance of stock markets.

In yet another embodiment, the present invention provides a method wherein the use of an EQL will find applications in weather prediction, warning and hazard. This includes but not limited to providing robust projections on amount of rainfall in a season, probability of drought, flood, Fire, frequency and strength of hurricanes.

In yet another embodiment, the present invention provides a method wherein the use of an EQL will find applications in the field of power distribution and management. The code will be very effective to projecting the efficient distribution network in keeping in mind future growth in demand.

In yet another embodiment, the present invention provides a method wherein the use of an EQL will find applications in urban development and planning. The EQL will provide the optimal design for proper organization of city blocks.

In another embodiment, the present invention provides a method wherein the use of an EQL will find applications in animal conservation and implementation of bio-conservation. This will also include but not limited to factoring out the future risk of population expansion and possible encroachment in the forest area.

In another embodiment, the present invention provides a method wherein the use of an EQL will find application in real state planning and development of algorithm could predict future prices based on current indicators.

In yet another embodiment, the present invention provides a method wherein the use of an EQL will find applications in retail market. The algorithm could project the prices for customers, profitability of the owners based on current supply and demand. It can help business owners to design and launch a brand, which can give best profit as well as customer satisfaction.

In another embodiment, the present invention provides a method wherein the use of an EQL will find applications in hospitality business. The EQL can provide business owners in ventures to invest and make profits.

In another embodiment, the present invention provides a method wherein the use of an EQL will find applications in aviation industry. It has the capability to project new routes, which will have high probability to yield huge profits.

In yet another embodiment, the present invention provides a method wherein the use of an EQL will find applications in efficient human resources management.

In yet another embodiment, the present invention provides a method wherein the use of an EQL will find applications in water distribution system. The EQL algorithm provides very effective way of distribution and sewerage of fresh water. The algorithm will be effective tool for fresh water conservation and management. It will provide an effective layout of pipes and junction to minimize water loss while maintaining effective pressure gradient.

In another embodiment, the present invention provides a method wherein the use of an EQL will find applications in distribution and effective use in renewable wind energy. The Algorithm will have ability to predict effective distribution of wind turbine within a limited area with an added feature for future expansion. Seasonal direction and speed of wind, spacing between the turbine and potential increase in turbine number will all be taken into consideration.

In another embodiment, the present invention provides a method wherein the use of an EQL will find applications in naval seasonal navigation.

In yet another embodiment, the present invention provides a method wherein the use of an EQL will find application in effective satellite navigation and collision avoidance form space debris. An ensemble map to space debris will allow EQL to extrapolate their most probable trajectories. The Satellites could be placed in such a way that probability of collision is minimized. This is very important for geo-stationary communication satellites, which are placed in near earth orbit and probability of collision with space debris is very high.

In yet another embodiment, the present invention provides a method wherein the use of an EQL will find effective use in risk factor management in metropolitan cities. Analysis of accident rates on an hourly basis, using covariates like population density and average vehicle density, the EQL algorithm can effectively provide a framework for traffic control layout for smooth traffic flow even during rush hours.

In yet another embodiment, the present invention provides a method wherein the use of an EQL like other regression and extrapolation model provide effective way to reduce dimensionality of the problem. It can very effectively find variables and components, which affect the system much more than other variables that have smaller effect. Advantageously, such reduction in dimensionality reduces complexity of the system.

In another embodiment, the present invention provides a method wherein the use of EQL will find an effective use in controlling and predicting a 3-dimensional protein only based on the amino acid sequence. The unique feature of the algorithm according to the invention is giving propensity of a 3D-fold based to a sequence. The EQL network will not only provide secondary structure prediction but also provide propensity for a 3D-fold, like EF-hand or beta barrel.

In another embodiment, the present invention provides a method, which can effectively predict an intrinsically disordered sequence that will have high propensity of aggregation.

In another embodiment, the present invention provides a method wherein the use of an EQL can work on the functional relationship between a genotype with a phenotype. Getting a functional relationship between activities of various genes and its phenotypical effects. The EQL can predict a new knock out which will give the desired outcome. In gene-therapy it can aid the physicians immensely by accurately predicting gene and functional relationship for a disorder.

In another embodiment, the present invention provides a method wherein the use of an EQL will find effective use in forest fire prevention. The EQL algorithm by projection of future weather condition could predict the most probable location of a forest fire.

In another embodiment, the present invention provides a method wherein the use of an EQL will find effective use in the field of astronomy and space science. Using the elemental characteristics of various stars, the EQL based algorithm could effectively predict regions in our galaxy where we can find extra-solar earth-like planets. The present disclosure will now be described more fully hereinafter with reference to the accompanying drawings, in which some exemplary embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternative are mutually exclusive, although the disclosure supports a definition that refers to only alternatives and “and/or.”

The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the terms “substantially,” “approximately,” and “about” may be substituted with “within [a percentage] of” what is specified, where the percentage includes 0.1, 1, 5, and 10 percent.

Throughout this application, the term “about” is used to indicate that a value includes the standard deviation of error for the device or method being employed to determine the value.

Further, a calculation and/or a method that is configured in a certain way is configured in at least that way, but it can also be configured in other ways than those specifically described.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps but is not limited to possessing only those one or more steps.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Any embodiment of any of the apparatuses, systems, and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

To the extent that any specific disclosure in the aforementioned references or other literature may be considered to anticipate any generic aspect of the present invention, the disclosure of the present invention should be understood to include a proviso or provisos that exclude of disclaim any such species that were previously disclosed. The aspects of the present invention, which are not anticipated by the disclosure of such literature, are also nonobvious from the disclosure of these publications, due at least in part to the unexpectedly superior results disclosed or alleged herein.

Below, the presently disclosed invention will be further described by way of examples, which are provided for illustrative purposes only and accordingly are not to be construed as limiting the scope of the invention.

### EXAMPLES OF APPLICATION OF EQL IN SEVERAL SYSTEMS

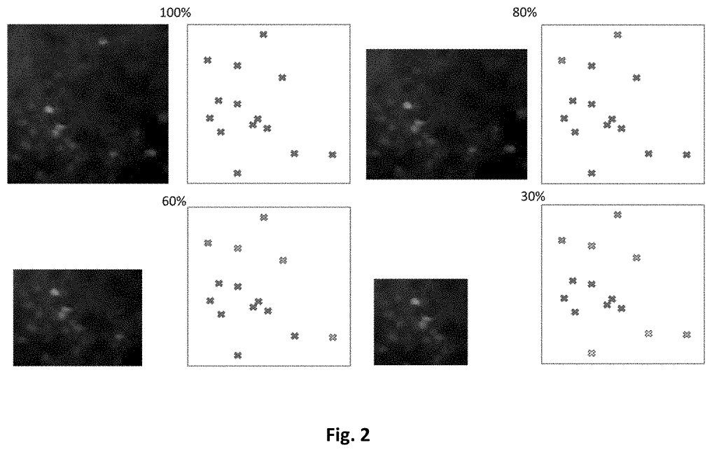

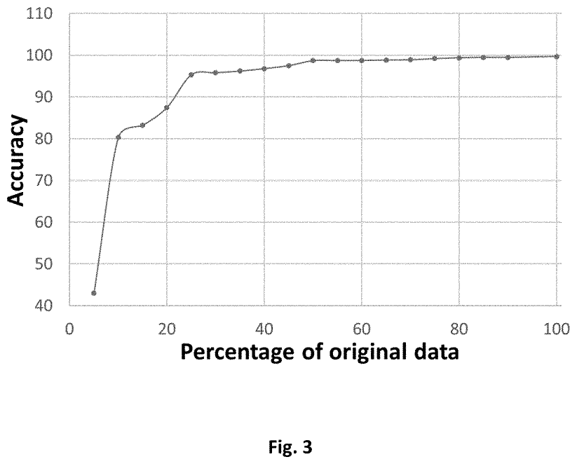

Example 1: A Reconstruction of visual cortical activity pattern with substantially reduced FOV. Using a genetically encoded calcium indicator (GCaMP6), the activity patterns of the primary visual cortex in mice upon patterned visual stimulation was recorded. The EQL network predicted the location of spiking neurons in visual cortex with 100% accuracy. The FOV was gradually reduced. The EQL algorithm was able to completely reconstruct the neuronal excitation profile even when 30% of the original FOV was available (FIG. 2). A complete performance profile of EQL with respect to reconstructed data is shown in FIG. 3. This demonstrated that the EQL algorithm has high efficiency in data extrapolation. We also tested other AI based extrapolation techniques like radial basis function (RBF) and demonstrated that EQL significantly outperforms the RBF (Table 2).

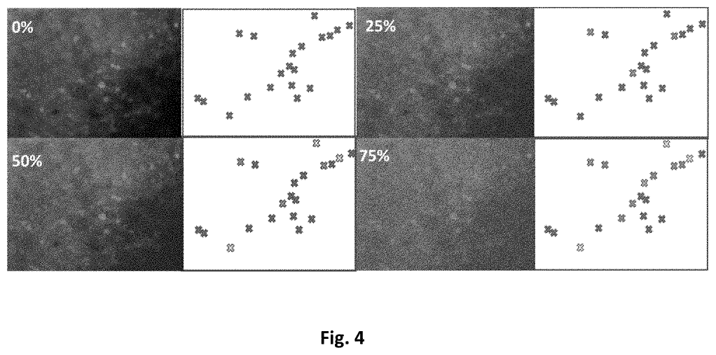

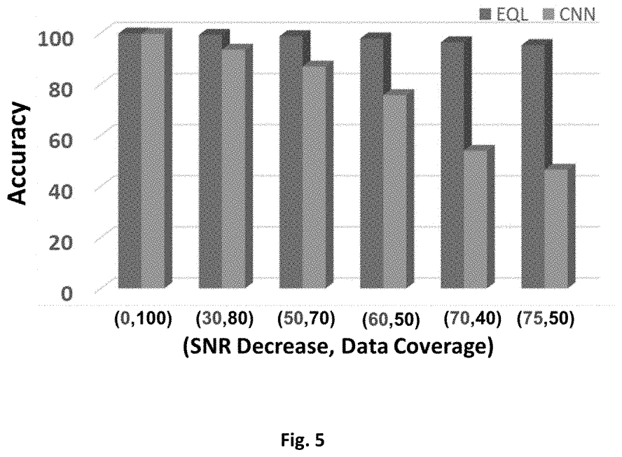

Example 2: EQL reconstructed neural activity data with decreased SNR: Disruption in neural interface results in significant decrease in SNR. To demonstrate our EQL algorithm's ability to perform at very low SNR level, Gaussian noise to the recorded visually stimulated cortical activity patterns was added (FIG. 4). EQL was completely able to reconstruct the visual cortical activity patterns even when the SNR is reduced by 75%. The detailed performance of the present invention method under various SNR conditions is listed in Table 2. Also tested was the efficiency of EQL by simultaneously decreasing FOV along with noise addition. Even in such a situation, EQL shows very high efficiency (FIG. 5).

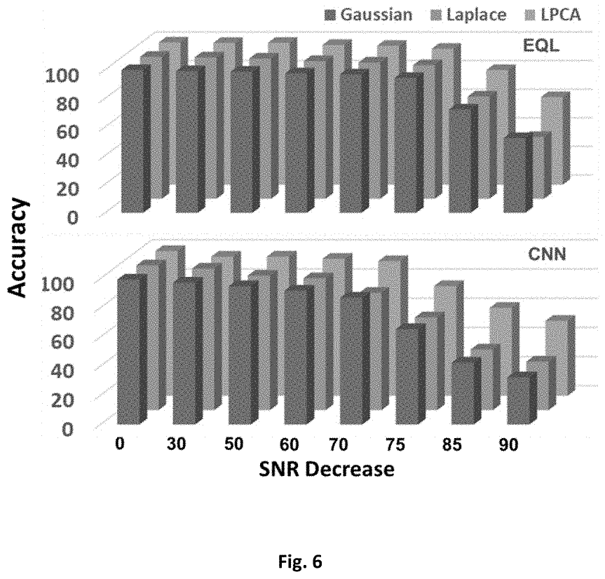

Example 3: Comparative advantage of EQL over CNN in accuracy and speed of reconstruction for neural activity data. In comparison with other AI based algorithms, like convolution neural network (CNN), the present invention EQL shows significant accuracy with reduced computational time (FIG. 5, Table 2) in reconstructing the noisy/lost neural activities/channels. The inventors further denoised the data using various filters and compared the efficiency between EQL and CNN. As clearly shown in FIG. 6, EQL outperforms CNN in all aspects.

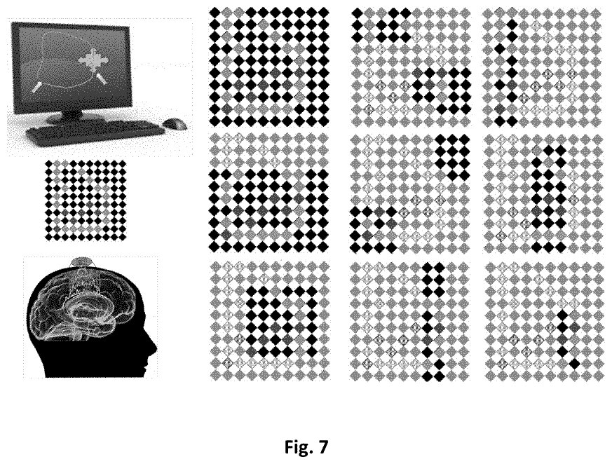

Example 4: A mouse cursor experiment was done for complete motion-signal reconstruction using an EQL network and data was captured in a 10×10 array (FIG. 7). In this experiment, a mouse in the screen was moved between two positions by 3 different pathways. The full signal reconstruction is carried out by use of the EQL network. Then, subsequently the inventors randomly intentionally made patches in the array faulty and reconstructed the whole signal using the EQL network. With only 20% of the data present, the EQL network was able to completely reconstruct all 3 pathways.

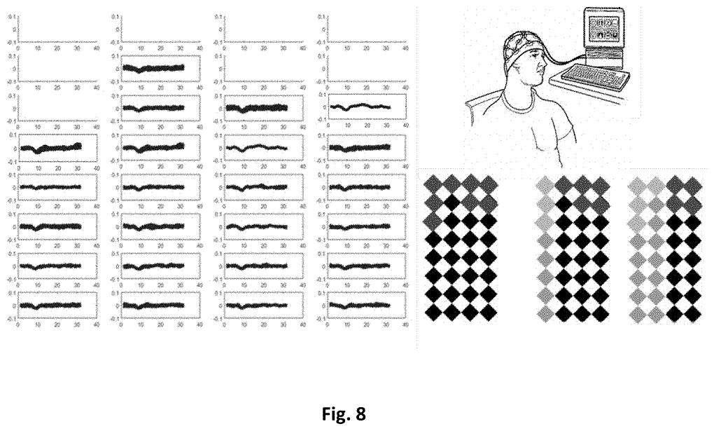

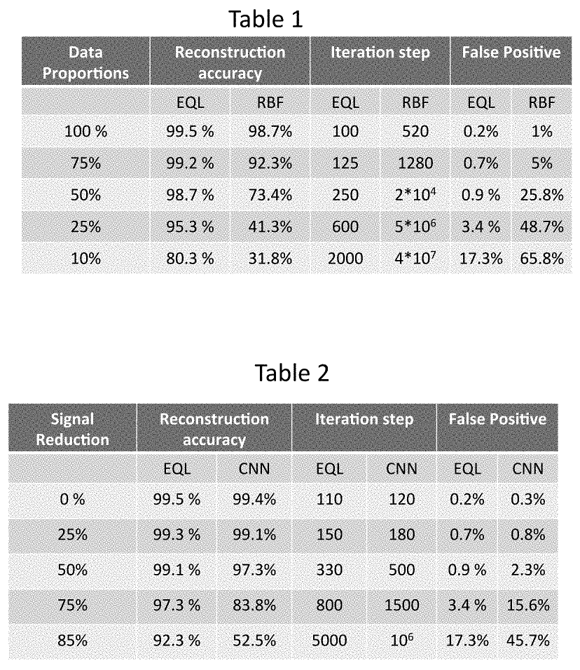

Example 5: A real-life 32 (8×4) channel array electrical-signal reconstruction was conducted using an EQL network. The inventors intentionally randomly made various block elements in the array faulty. Electrical data generated from a stimulus is shown on the left panels. The inventors then extrapolated the remaining signals with EQL network pattern. With only 50% of the array signal present, the inventors could back-project the entire data set as shown in FIG. 8 (Right panels). The translucent area in the array represents reconstructed signals from EQL network pattern.

As used in this document, both in the description and in the claims, and as customarily used in the art, the words “substantially,” “approximately,” and similar terms of approximation are used to account for tolerances, variations, and imprecisions that are inescapable parts of fabricating any mechanism or structure in the physical world, and of performing a method in the physical and/or digital world.

While the invention has been described in detail, it will be apparent to one skilled in the art that various changes and modifications can be made and equivalents employed, without departing from the present invention. It is to be understood that the invention is not limited to the details of construction, the arrangements of components, and/or the method set forth in the above description or illustrated in the drawings. Statements in the abstract of this document, and any summary statements in this document, are merely exemplary; they are not, and cannot be interpreted as, limiting the scope of the claims. Further, the figures are merely exemplary and not limiting. Topical headings and subheadings are for the convenience of the reader only. They should not and cannot be construed to have any substantive significance, meaning or interpretation, and should not and cannot be deemed to indicate that all of the information relating to any particular topic is to be found under or limited to any particular heading or subheading. The purpose of the Abstract of this document is to enable the U.S. Patent and Trademark Office, as well as readers who are not familiar with patent or legal terms or phraseology, to determine quickly from a cursory inspection the nature and essence of the technical disclosure of the application. The Abstract is not intended to define the invention, nor is it intended to limit to the scope of the invention. Therefore, the invention is not to be restricted or limited except in accordance with the following claims and their legal equivalents.

### REFERENCES

The following references, to the extent that they provide exemplary procedural or other details supplementary to those set forth above, are specifically incorporated by reference.

1. Gustafson S C, Little G R and Simon D M, (1990) Neural network for interpolation and extrapolation. SPIE, Applications of Artificial Neural Networks,1294:

2. Sahoo S S, Lampert C H and Martius G, Learning Equations for Extrapolation and Control, arXiv:1806.07259v1 [cs.LG] 19 Jun. 2018.

3. Zhang W, (1992) Shift-Invariant Neural Network for Image Processing: Learning and generalization SPIE. 1709:258

4. Krizhevsky A, Sutskever I, and Hinton GE, (2012) Imagenet classification with deep convolutional neural networks, Advances in Neural Information Processing Systems. 1: 1097-1105.

5. Hettiarachchi P, Hall M J, and Minns A W. (2005). The extrapolation of artificial neural networks for the modelling of rainfall-runoff relationships. Journal of Hydroinformatics. 7 (4): 291-296.

6. Schmidt M and Lipson H, (2009) Distilling free-form natural laws from experimental data. Science, 324(5923):81-85.

7. Schwenker F, Kestler H A, Palm G, (2001). Three learning phases for radial-basis-function networks. Neural Networks. 14: 439-458.

8. Buhmann M D, (2009). Radial Basis Functions: Theory and implementations. Cambridge University.

9. Wessberg J, Stambaugh C R, Kralik J D, Beck P D, Laubach M, Chapin J K, Kim J, Biggs S J, Srinivasan M A and Nicolelis M A. (2000). Real-time prediction of hand trajectory by ensembles of cortical neurons in primates. Nature. 408 (6810): 361-365

10. Lauritzen S L, (1981). Time series analysis in 1880. A discussion of contributions made by T. N. Thiele. International Statistical Review. 49 (3): 319-331.

11. Chuang A T, Margo C E, Greenberg P B, (2014). Retinal implants: a systematic review. The British Journal of Ophthalmology. 98 (7): 852-856.

12. Alexander R, Macknik S, Martinez-Conde S, (2018). Microsaccade Characteristics in Neurological and Ophthalmic Disease. Frontiers in Neurology. 9 (144): 144.

13. Berg K T, Nelson B, Harrison A R, McLoon L K, and Michael S, (2010). Pegylated Interferon Alpha-Associated Optic Neuropathy. Journal of Neuro-Ophthalmology. 30 (2): 117-22

14. Carelli V, Ross-Cisneros F N, Sadun A A, (2004). Mitochondrial dysfunction as a cause of optic neuropathies. Progress in Retinal and Eye Research. 23 (1): 53-89.

15. Miller N R, Newman N J , Valerie B, Kerrison J B. (2007) Walsh & Hoyt's Clinical Neuro-Ophthalmology: The Essentials. Lippincott Williams & Wilkins.

16. Grill W M, Norman S E, and Bellamkonda R V (2009). Implanted neural interfaces: biochallenges and engineered solutions. Annual review of biomedical engineering, 11, 1-24.

17. Geary J, (2002). The Body Electric. Rutgers University Press. p. 214. ISBN 9780813531946.

18. Navarro X, Krueger T B, Lago N, Micera S, Stieglitz T, and Dario P, (2005). A critical review of interfaces with the peripheral nervous system for the control of neuro-prostheses and hybrid bionic systems. Journal of the peripheral nervous system: 10(3), 229-258.

19. Varga M, Luniak M, and Wolter K J,(2013) Novel self-folding electrode for neural stimulation and recording, Electronics and Nanotechnology (ELNANO), IEEE XXXIII International Scientific Conference, Kiev, 237-240.

What is claimed is:

1. A method comprising the steps of:
providing known input and output training data;
determining an underlying mathematical equation based on the relationship between the known input and output training data;
defining the equation learning (EQL) algorithm by multiple iterations of the underlying mathematical equation by minimization of error in defining the relationship between the input and output training data;
receiving a number of set of inputs;
processing the set of inputs using the equation learning (EQL) algorithm to generate extrapolated or compressed output data points from the set of inputs;
processing one or more of the extrapolated or compressed output data points to generate at least one output for each of a set of inputs;
wherein the equation learning (EQL) algorithm uses a many-to-one function to compress the data;
wherein the redundancy of the set of inputs is reduced by the equation learning (EQL) algorithm by establishing key variables and components that affect the at least one output, thereby allowing prediction of the at least one output from a fewer number of set of inputs.

2. The method according to claim 1, wherein the EQL algorithm compensates for data loss in faulty and aging neural interfaces and increases the longevity of neuro-prosthetics by data compression up to 10,000 folds and by extrapolation, so that the data can be efficiently transmitted through the neural interfaces with low power consumption.

3. The method according to claim 1, wherein the EQL algorithm is applied for biotelemetry;
assisting people with prosthetic arms/legs via neuronal interfaces controlling their motions and in neuro-degenerative diseases comprising Parkinson's by characterizing the uncoordinated motion, and to give stimulation feedback to correct the motion.

4. The method according to claim 1, wherein the EQL algorithm is applied to recognize the signal pattern and generate feedback to reduce pain, improve memory in neurodegenerative diseases.

5. The method according to claim 1, wherein the EQL algorithm is used for analysis of the rate of blood flow in the heart valves and determination of the probability of stroke, arrhythmia etc.

6. The method according to claim 1, wherein EQL algorithm is applied for predicting the structure of three-dimensional proteins based in an amino acid sequence and establishing a functional relationship between a genotype with a phenotype by predicting mutations.

7. The method according to claim 1, wherein the EQL algorithm is applied in monitoring performance of stock/consumer markets by analyzing the temporal variation of price and changes in volume of transactions.

8. The method according to claim 1, wherein the EQL algorithm is used for optimizing power distribution network, urban development planning and population expansion.

9. The method according to claim 1, wherein the EQL algorithm is applied in traffic signaling in surface, water and air aviation to project new routes, satellite navigation, and collision avoidance.

10. The method according to claim 1, wherein the EQL algorithm is used for at least one selected from the group consisting of: management of human resources, water resources, and distribution and sewerage of fresh water.

11. The method according to claim 1, wherein the EQL algorithm features employ the combination of an image recognition-based feature extraction, optionally shape and/or size, to classify aspects of image recognition from a relatively small amount of training data.

12. The method according to claim 11, wherein the EQL algorithm is applied for disease detection including sickle cell anemia, thalassemia, and blood cancer comprising hematological malignancies including but not limited to Acute lymphoblastic leukemia, Acute myelogenous leukemia, Chronic lymphocytic leukemia, Chronic myelogenous leukemia, Acute monocytic leukemia, Hodgkin's lymphomas and Non-Hodgkin's lymphomas.

13. The method according to claim 11, wherein the EQL algorithm is used for image detection, segmentation and classification to detect pathogenic bacteria and microbes present in the blood samples.

14. The method according to claim 11, wherein the EQL algorithm is trained to identify images of toxic oligomers and fibrils in cerebrospinal fluid (CSF) and to detect and diagnose neurodegenerative diseases comprising Alzheimer's, Parkinson's and Huntington's disease.

15. The method according to claim 11, wherein the EQL algorithm is applied for efficient image segmentation and classification to measure the pollutant population in a sea and fresh water system, thus determining oil spills and its effect on environment.

16. The method according to claim 11, wherein the EQL algorithm is used for weather prediction, warning and hazards that include but not limited to providing robust projections on amount of rainfall in a season, probability of drought, flood, Fire, and frequency and strength of hurricanes.

17. The method according to claim 11, wherein the EQL algorithm is applied for management of human resources, water resources, and distribution and sewerage of fresh water.

18. The method according to claim 11, wherein EQL algorithm is used in the field of astronomy and space science for locating extrasolar earth-like planets from the transit images and elemental characteristics of stars.
